(19) United States
(12) Patent Application Publication
(10) Pub. No.: US 2023/0165589 A1
(43) Pub. Date: Jun. 1, 2023

(54) DEVICES, SYSTEMS, AND METHODS FOR PYLORIC OCCLUSION

(71) Applicant: BOSTON SCIENTIFIC SCIMED, INC., Maple Grove, MN (US)

(72) Inventors: Martyn G. Folan, Galway (IE); Michael Walsh, Galway (IE); Gary Gilmartin, Foxford (IE); Daniel Tuck, Galway (IE); Martin Murray, Loughrea (IE)

(73) Assignee: BOSTON SCIENTIFIC SCIMED, INC., Maple Grove, MN (US)

(21) Appl. No.: 17/992,794

(22) Filed: Nov. 22, 2022

Related U.S. Application Data
(60) Provisional application No. 63/283,758, filed on Nov. 29, 2021.

(51) Int. Cl. A61B 17/12

(52) U.S. Cl. CPC A61B 17/1219; A61B 17/1204; A61B 17/12099

(57) ABSTRACT

An occlusion device having a support structure and an expandable occlusion element associated with the support structure. The expandable occlusion element is formed of a self-expanding material, such as a hydrogel. A retainer may be provided to hold the expandable occlusion element with respect to the support structure of the occlusion device, within or outside the support structure, and positioned such that expansion of the expandable occlusion element occludes a lumen through the support structure and the occlusion device. Expansion of the expandable occlusion element may be reversible, or the expandable occlusion element may be releasable from the support structure.

### CROSS-REFERENCE TO RELATED APPLICATIONS

This application claims the benefit of priority of U.S. Provisional Application No. 63/283,758, filed Nov. 29, 2021, the entire disclosure of which is hereby incorporated by reference herein for all purposes.

### FIELD

The present disclosure relates generally to the field of devices, systems, and methods for occluding a body passage. More particularly, the present disclosure relates to devices, systems, and methods for reversibly occluding a body passage. Even more particularly, the present disclosure relates to devices, systems, and method for reversibly occluding a body passage in the gastrointestinal tract, such as the pylorus.

### BACKGROUND

Currently there exists a category of medical devices within the gastrointestinal (GI) discipline known as obstruction devices. These devices are designed for obstructing or reducing flow through a body lumen, in particular for obstructing or reducing flow of gastric contents across the pyloric valve (pylorus). Such devices may be known as a pyloric plug or just a “plug”. The plug is particularly useful to stop the flow of stomach contents to the proximal gut (small intestine) which includes the duodenum and the initial part of the jejunum. Such a need arises, for example, after creating an alternative path of flow through a gastro-jejunum or similar anastomosis which bypasses the proximal gut. Other instances in which obstruction or reduction of flow to the proximal gut exist, such as after surgery in the duodenal region or in the pancreas or bile outputs to the duodenum.

Various pyloric devices have become available in the industry. However, there is still a need for improvements to pyloric plugs, such as pyloric plugs which function with natural anatomical functions to achieve full closure yet which may be reversible.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various principles of the present disclosure, an occlusion device includes a support structure having a lumen defined therethrough, an expandable occlusion element formed separately from the support structure and held with respect to the support structure, where the expandable occlusion element is expandable with respect to the support structure to occlude the support structure lumen.

In some embodiments, the expandable occlusion element is formed from a self-expanding material. In some embodiments, the expandable occlusion element is formed from a hydrogel. In some embodiments, the expandable occlusion element is held within a retainer associated with the support structure. In some embodiments, the retainer is formed of a porous or permeable material.

In some embodiments, the expandable occlusion element is held within a retainer associated with the support structure and the retainer includes a disruptor element configured to disrupt the integrity of the retainer to release the expandable occlusion element therefrom.

In some embodiments, the expandable occlusion element is held within a retainer associated with the support structure and the retainer is separately formed from the support structure. In some embodiments, the retainer is deployed separately from the support structure. In some embodiments, the support structure is configured to shift between an unexpanded deployment configuration and an expanded deployment configuration, and to draw the retainer into the support structure lumen upon expanding into the deployment configuration.

In some embodiments, expansion of the expandable occlusion element is reversible to allow passage of materials through the support structure lumen. In some embodiments, the occlusion device further includes a secondary scaffold configured to deliver an element to the expandable occlusion element which causes reduction of the size of the expandable occlusion element.

In accordance with various principles of the present disclosure, a system for occluding a body passage includes a delivery device, and an occlusion device, the occlusion device having a support structure with a lumen defined therethrough, and an expandable occlusion element formed separately from the support structure and held with respect to the support structure, where the expandable occlusion element is expandable with respect to the support structure to occlude the support structure lumen.

In some embodiments, the system where the expandable occlusion element is formed from a self-expanding material. In some embodiments, the expandable occlusion element is formed from a hydrogel.

In some embodiments, the expandable occlusion element is held within a retainer associated with the support structure of the occlusion device of the system.

In accordance with various principles of the present disclosure, a method of occluding a body passage includes delivering an occlusion device to a body passage in a delivery configuration; deploying the occlusion device so that a support structure of the occlusion device shifts to an expanded deployment configuration; and causing an expandable occlusion element formed separately from the support structure to expand to at least partially occlude a lumen extending through the support structure.

In some embodiments, causing the expandable occlusion element to expand includes exposing the expandable occlusion element to an expansion-inducing condition.

In some embodiments, the method further includes reversing the expansion of the expandable occlusion element to allow materials to pass through the support structure lumen.

In some embodiments, the method further includes release of the expandable occlusion element from the support structure to allow materials to pass through the support structure lumen.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a schematic representation of a portion of a gastrointestinal system with an occlusion device, such as formed in accordance with various principles of the present disclosure, positioned across a pylorus.

FIG. 2 illustrates a schematic representation, in cross-section, of a delivery configuration of an occlusion device, such as formed in accordance with various principles of the present disclosure, within a delivery device.

FIG. 3A illustrates an elevational view, in cross-section, of an embodiment of an occlusion device formed in accordance with various principles of the present disclosure and in an expanded unoccluded configuration.

FIG. 3B illustrates an elevational view, in cross-section, of an embodiment of an occlusion device similar to that of FIG. 3A, but in an occluded configuration.

FIG. 4A illustrates an elevational view, in cross-section, of another embodiment of an occlusion device formed in accordance with various principles of the present disclosure and in an expanded unoccluded configuration.

FIG. 4B illustrates an elevational view, in cross-section, of an embodiment of an occlusion device similar to that of FIG. 4A, but in an occluded configuration.

FIG. 5A illustrates an elevational view, in cross-section, of another embodiment of an occlusion device formed in accordance with various principles of the present disclosure and in an expanded unoccluded configuration.

FIG. 5B illustrates an elevational view, in cross-section, of an embodiment of an occlusion device similar to that of FIG. 5A, but in an occluded configuration.

FIG. 6A illustrates an elevational view, in cross-section, of another embodiment of an occlusion device formed in accordance with various principles of the present disclosure and in an expanded unoccluded configuration.

FIG. 6B illustrates an elevational view, in cross-section, of an embodiment of an occlusion device similar to that of FIG. 6A, but in an occluded configuration.

FIG. 6C illustrates an end view of an occlusion device as illustrated in FIG. 6A.

FIG. 6D illustrates an end view of an occlusion device as illustrated in FIG. 6B.

FIG. 7A illustrates an elevational view, in cross-section, of another embodiment of an occlusion device formed in accordance with various principles of the present disclosure and in an expanded unoccluded configuration.

FIG. 7B illustrates an elevational view, in cross-section, of an embodiment of an occlusion device similar to that of FIG. 7A, but in an occluded configuration.

FIG. 8A illustrates an elevational view, in cross-section, of another embodiment of an occlusion device formed in accordance with various principles of the present disclosure and in an expanded unoccluded configuration.

FIG. 8B illustrates an elevational view, in cross-section, of an embodiment of an occlusion device similar to that of FIG. 8A, but in an occluded configuration.

FIG. 9A illustrates an elevational view, in cross-section, of another embodiment of an occlusion device formed in accordance with various principles of the present disclosure and in an expanded unoccluded configuration.

FIG. 9B illustrates an elevational view, in cross-section, of an embodiment of an occlusion device similar to that of FIG. 9A, but in an occluded configuration.

FIG. 10A illustrates an elevational view, in cross-section, of another embodiment of an occlusion device formed in accordance with various principles of the present disclosure and in an expanded unoccluded configuration.

FIG. 10B illustrates an elevational view, in cross-section, of an embodiment of an occlusion device similar to that of FIG. 10A, but in a collapsed delivery configuration.

FIG. 10C illustrates an elevational view, in cross-section, of an embodiment of an occlusion device similar to that of FIG. 10A and FIG. 10B, transitioning from the delivery configuration of FIG. 10B to the deployed configuration of FIG. 10A.

FIG. 11A illustrates an elevational view, in cross-section, of another embodiment of an occlusion device formed in accordance with various principles of the present disclosure and in an expanded unoccluded configuration.

FIG. 11B illustrates an elevational view, in cross-section, of an embodiment of an occlusion device similar to that of FIG. 11A, but in an occluded configuration with expandable elements contributing to the occluded configuration beginning to be released to return the occlusion device to an unoccluded configuration.

FIG. 11C is a cross-sectional view along line XI-XI of FIG. 11A.

FIG. 12A illustrates an elevational view, in cross-section, of another embodiment of an occlusion device formed in accordance with various principles of the present disclosure and in an expanded occluded configuration.

FIG. 12B illustrates an elevational view, in cross-section, of an embodiment of an occlusion device similar to that of FIG. 12A, but beginning to be shifted into an unoccluded configuration.

FIG. 12C illustrates an elevational view, in cross-section, of an example of an embodiment of an occlusion device similar to that of FIG. 12A and FIG. 12B, with a device passing therethrough.

FIG. 13A illustrates an elevational view, in cross-section, of another embodiment of an occlusion device formed in accordance with various principles of the present disclosure and in an expanded occluded configuration.

FIG. 13B illustrates an elevational view, in cross-section, of an embodiment of an occlusion device similar to that of FIG. 13A, but with a lumen-opening device beginning to open the lumen through the occlusion device.

FIG. 13C illustrates an elevational view, in cross-section, of an embodiment of an occlusion device similar to that of FIG. 13B, with the opening device further advanced to open the lumen through the occlusion device and with a device passing through the lumen.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore.

In accordance with various principles of the present disclosure, an occlusion device is delivered and deployed through a body passage, the occlusion device including a support structure and a separate expandable occlusion element associated therewith. It will be appreciated that reference may be made herein to terms such as occlude, obstruct, block, and other grammatical forms thereof, such terms being used interchangeably herein without intent to limit. It will further be appreciated that terms such as deploy, extend, place, etc., and various grammatical forms thereof, may be used interchangeably herein without intent to limit, with respect to a device as disclosed herein. Moreover, reference may be made to placement of a device through, across, in, etc., such terms being used interchangeably herein without intent to limit. Finally, it will be appreciated that terms such as lumen, passage, etc. may be used interchangeably herein without intent to limit. In some embodiments, the body passage is a pylorus. In some embodiments, the occlusion device is delivered with the assistance of a delivery device such as a flexible elongate member (e.g., catheter, endoscope with a working channel therethrough for delivery of devices, etc.). For instance, the occlusion device may be delivered through a working channel of a scope with a catheter-based delivery system by direct vision and/or fluoroscopy. In some embodiments, the occlusion device may be delivered independently of a scope using fluoroscopy. Additionally or alternatively, the occlusion device may be delivered over a guide wire. The occlusion device (or at least the support structure thereof) may be delivered in a delivery configuration configured to facilitate delivery to a treatment site, and shiftable into a deployment configuration, configured to facilitate deployment at a deployment site. In the delivery configuration, the occlusion device may be in a collapsed and/or unexpanded configuration within a lumen of the delivery device. In the deployment configuration, the occlusion device may be in an expanded configuration, such as to be securely deployed at the deployment site. In some embodiments, when in the delivery configuration, the occlusion device (or at least the support structure thereof) is contracted in diameter (e.g., to fit within the delivery device) and extended in length. In some embodiments, when in the delivery configuration, the occlusion device contacts the tissue at the deployment site to be securely positioned with respect to the tissue.

The support structure of an occlusion device formed in accordance with various principles of the present disclosure may be in the form of a scaffolding structure configured to support a separate expandable occlusion element. For the sake of convenience, and without intent to limit, the scaffolding structure may be referenced herein as a scaffold and/or a stent. In some embodiments, the stent is configured to be placed in a body passage and has a lumen therethrough. The stent may be formed of a metal, a polymer, or a combination thereof. The stent may be configured to shift between a delivery configuration in which the stent is configured (e.g., collapsed, constrained, unexpanded, etc.) to facilitate delivery through a body passage (e.g., transluminally, transcatheterally, etc., such as through potentially tortuous body passages rather than by open surgery), and a delivery configuration (e.g., expanded) in which the stent is configured to facilitate deployment at a deployment site, such as by contacting the walls of the body passage in which the stent is placed. The stent may be self-expanding, or may be expanded with the assistance of another device (e.g., an expandable balloon). Examples of stents include, without limitation, stents having one or more strut members combined and/or arranged to form a rigid and/or semi-rigid stent structure. For example, the strut members may be formed of one or more wires or filaments which are braided, wrapped, intertwined, interwoven, weaved, knitted, looped (e.g., bobbinet-style), knotted, or the like to form a scaffold configuration. Alternatively, the stent may be a monolithic structure formed from a cylindrical tubular member, such as a single, cylindrical tubular laser-cut tubular member, in which the remaining portions of the tubular member form the strut members. The strut members of a self-expanding stent may be formed of shape-memory material, such as Nitinol or Elgiloy, so that the stent returns to a pre-shaped expanded configuration from a collapsed configuration. Openings or interstices may be defined between adjacent strut members.

A support structure formed in accordance with various principles of the present disclosure may be formed with a saddle region configured to extend through a body passage, and a retention member extending radially outwardly from one or both ends of the saddle region. The retention members may alternately be referenced as flanges without intent to limit. The retention members may be formed integrally with or separately from the saddle region. In some embodiments, one or both of the retention members are double-wall retention members, the double walls of each retention member being adjacent or spaced apart from each other. In some embodiments, more than one retention member may extend radially from one or each end of the saddle region, adjacent or spaced apart from each other.

Examples of embodiments of support structures which may be used to advantage in an occlusion device formed in accordance with various principles of the present disclosure include stents such as disclosed in one or more of the following published patent applications, each of which is incorporated by reference herein in its entirety for all purposes: U.S. Patent Application Publication US2009/0281557, filed Apr. 21, 2009, titled Tissue Anchor For Securing Tissue Layers, and published Nov. 12, 2009; U.S. Patent Application Publication US2018/0280166, filed Mar. 29, 2018, titled Stents With Dual Tissue-Wall Anchoring Features, and published Oct. 4, 2018; and U.S. Patent Application Publication US2019/0298559, filed Mar. 22, 2019, titled Device, Systems, And Methods For Pyloric Occlusion, and published Oct. 3, 2019.

Various regions of the support structure may include tissue ingrowth promoting regions, such as uncovered and/or bare metal portions exposed to the tissue at the deployment site. As may be appreciated by those of ordinary skill in the art, a support structure in the form of a stent may have interstitial spaces in which tissue may grow, thereby securing the support structure at the deployment site. As may be further appreciated by those of ordinary skill in the art, a coating may be applied to the support structure to affect the interaction of the material of the support structure with adjacent tissue. For instance, in some embodiments, a coating may be applied to at least a portion of the support structure to inhibit or prevent tissue ingrowth with respect to the support structure (such as tissue ingrowth into the structure of the support structure, such as into interstices or openings therein), which may facilitate removal of the support structure if desired or medically indicated. Such coating may alternatively be referenced as a liner without intent to limit. The coating may be elastomeric or non-elastomeric. The coating may be formed of any of a variety of biocompatible materials such as a polymeric material, including, without limitation, polyethylene terephthalate (PET), silicone, polyurethane, etc. Examples of coatings for support structures which may be used to advantage in an occlusion device formed in accordance with various principles of the present disclosure include coatings disclosed in one or more of the following published patent applications and patents, each of which is incorporated by reference herein in its entirety for all purposes: U.S. Patent Application Publication US2017/0071767, filed Sep. 9, 2016, titled Stent With Coated Struts, and published Mar. 16, 2017; U.S. Patent Application Publication US2018/0250118, filed Mar. 1, 2018, titled Esophageal Stent Including An Inner Liner, and published Sep. 6, 2018; U.S. Patent Application Publication US2019/0298559, filed Mar. 22, 2019, titled Devices, Systems, And Methods For Pyloric Occlusion, and published Oct. 3, 2019; U.S. Patent Application Publication US2021/0015598, filed Jul. 16, 2020, titled Stents, Systems, And Methods For Gastrointestinal Tract Treatment, and published Jan. 21, 2021; and U.S. Pat. No. 10,779,967 to Walsh et al., titled Stents With Dual Tissue-Wall Anchoring Features, and issued on Sep. 22, 2020.

An expandable occlusion element may be associated with the support structure, such as by being held with respect to the support structure, and/or by being coupled (directly or indirectly) to the support structure as a separate element, and/or otherwise being delivered with or to the support structure. The expandable occlusion element is associated with the support structure to adjust the size of the lumen through the support structure, such as by expanding to occlude the lumen through the support structure. It will be appreciated that terms such as associated with, positioned with respect to, etc. (and other grammatical forms thereof) may be used interchangeably herein without intent to limit to refer to a positional or structural relationship, such as holding, maintaining, retaining, etc. one structure with respect to another structure. The location of the expandable occlusion element with respect to the support structure may be selected to effect the desired obstructive/occlusive effect, and may be positioned inside and/or outside and/or along the length of and/or along a portion of and/or at an end of the support structure.

The occlusion device may include a retainer structure configured and positioned to hold the expandable occlusion element with respect to the support structure. For instance, a retainer may be provided with a wall element configured to retain the expandable occlusion element with respect to the support structure. It will be appreciated that terms such as hold, retain, carry, contain, constrain, etc., and other grammatical forms thereof, may be used interchangeably herein without intent to limit. The retainer defines a retention area in which the expandable occlusion element may be held. It will be appreciated that terms such as retention area, pouch, space, cavity, etc. may be used interchangeably herein without intent to limit. The retainer may form the retention area on its own or in conjunction with at least a portion of the support structure. In some embodiments, the retainer may be formed as a wall spaced apart from the support structure forming the retention area therebetween. For instance, the retainer may be formed from by a liner, such as formed from a material similar or the same as a coating applied to the support structure such as described above. In some embodiments, the retainer is formed during the same process during which the coating is applied to the support structure. For instance, a mandrel spaced apart from the support structure may be used to form the retainer with the material used to coat the support structure. The coating on the support structure may be taut with respect to the support structure, with the coating forming the retainer loose with respect to the support structure to form the retainer.

In some embodiments, the expandable occlusion element is a separate element which is coupled to and trails proximal to the support structure or leads the support structure (distal to the support structure). Alternatively, the separate expandable occlusion element is separately delivered to the support structure. For instance, the expandable occlusion element may be injected into a pouch formed by a retainer element after deployment of the occlusion device. Alternatively, the expandable occlusion element may be contained within a retainer delivered spaced apart from the ultimate position in which the expandable occlusion element is to expand. Upon deployment and expansion of the support structure, the retainer, which may be coupled with the support structure, may be pulled into place with respect to the support structure (e.g., within the lumen of the support structure) as the support structure is deployed (e.g., expanded), such as to occlude the lumen through the support structure. In some embodiments, the expandable occlusion element causes the structure of the support structure to expand or otherwise to be modified, such as to increase an occlusive effect and/or to secure the positioning of the occlusion device in place with respect to the treatment site.

In accordance with various principles of the present disclosure, the expandable occlusion element includes an expandable occlusion material is capable of self-expansion (the material itself expands), such as without addition thereto of another material and/or by a change in the structural configuration of the expandable occlusion element itself. The expandable occlusion element may be caused to expand in any of a variety of manners, depending on the nature and properties of the expandable occlusion element. For instance, the expandable occlusion material may change in size upon exposure to an expansion-inducing condition, such as upon reaction to environmental changes, such as in reaction to exposure to another element such as a liquid, and/or in reaction to a change in an environmental condition such as temperature or pH or pressure. The expandable occlusion element can be composed of homopolymers or copolymers, with a network structure and physical integrity created by the presence of cross-links, such as of a chemical (tie-points, junctions) and/or or a physical (entanglements, crystallites) nature. In some embodiments, the expandable occlusion material changes size as a result of molecular changes thereto. For instance, the expandable occlusion material may be a gel and/or hydrogel, such in the form of beads. As used herein, hydrogels are three-dimensional crosslinked hydrophilic polymers (e.g., a polymer network) that are able to swell in an aqueous environment (water or biological fluids) without dissolution, such as by absorbing large amounts of fluid such as water. Hydrogels remain insoluble due to the presence of crosslinks, which may be, for example, physical, chemical, or both. In some instances, the insolubility of the hydrogel is not permanent, and the particles biodisintegrate in vivo. Because of their high water affinity, environmental sensitivity, and high permeability, hydrogels have been widely used as a carrier for drug delivery systems and thus are considered suitable for use with implantable occlusion devices as disclosed herein. Various hydrophilic polymers can be used to produce hydrogels, including, without limitation: natural polymers, for example, alginate and carrageenan (anionic polymers), chitosan (cationic polymer), dextran, agarose, cellulose, and derivatives (neutral polymers), and pectin; synthetic polymers, for example, polyethylene glycol (PEG) and polylactic acid (PLA) (polyesters), acrylates, and polyvinyl alcohol (PVA); and/or combinations of natural and synthetic polymers, for example, alginate and Pluronics© (block copolymers PPOPEO-PPO). Hydrogels used as an expandable occlusion material in accordance with various principles of the present disclosure may be selected from suitable members of the following, among many others: homopolymers and copolymers of acrylic acid, methacrylic acid, acrylamides including N-alkylacrylamides, alkylene oxides such as ethylene oxide and propylene oxide, vinyl alcohol, vinyl pyrrolidone, ethylene imine, ethylene amine, acrylonitrile and vinyl sulfonic acid, amino acids such as lysine and glutamic acid and maleic anhydride, hydrophilic polyurethanes, proteins, collagen, cellulosic polymers such as methyl cellulose and carboxymethyl cellulose, dextran, carboxymethyl dextran, modified dextran, alginic acid, pectinic acid, hyaluronic acid, chitin, pullulan, gelatin, gellan, xanthan, starch, carboxymethyl starch, chondroitin sulfate, guar, and further copolymers, derivatives and mixtures of the foregoing. Many of these polymers may be physically crosslinked, chemically crosslinked, or both, to form hydrogels.

A hydrogel particle used in accordance with the present disclosure may undergo swelling in water such that its longest linear cross-sectional dimension (e.g., for a sphere, the diameter) increases by at least 5% or even more than 25%, including increments of 1% therebetween. A hydrogel particle, as defined herein, also embraces a particle that is capable of absorbing water in an amount such that the water constitutes at least 10% of the total weight of the particle. For instance, in accordance with various principles of the present disclosure, the expandable occlusion element may be in the form of a quantity of gel beads placed within a retention area defined by a retainer, the volume of beads depending on the degree of expansion capable for the selected beads if desired or medically indicated for the occlusion device. The selected beads may have the ability, on exposure to liquids (typically water), to expand to 200-500% their initial size. In some embodiments, commercially-available beads may be rated to expand over a period of 10 minutes, and may expand over a longer period, such as 48 hours. In some embodiments, a fast, relatively immediate, closure (e.g., 10 minutes) may be advantageous to ensure closure during the current intervention. A longer closure may be advantageous where there may be underlying anatomical considerations. For instance, a slow closure may allow the device to fit better with respect to underlying anatomical variations, and/or may allow for gastric pressure and peristaltic normalization with the freshly augmented gastric arrangement, and/or may allow for normalization of resident gaseous and nutritional content within the initial GI tract. Similar to a self expanding GI stent, an occlusion device formed in accordance with various principles of the present disclosure may take approximately 48-72 hours to reach its full diameter, as a sudden expansion may cause a vessel perforation or rupture. A relatively slow expansion allows for anatomical relief and vessel stretching. It will be appreciated that expansion may be initiated upon exposure to fluids (e.g., body fluids, and or introduced fluids such as water), and/or body temperature or other environmental factors at the deployment site.

The expandable occlusion material may be formed by sol-gel processing such as described at https://www.twi-global.com/technical-knowledge/faqs/faq-what-is-sol-gel-processing. Such process involves agglomeration of micro particles or molecules in a solution (sols) agglomerate and under controlled conditions to eventually link together to form a coherent network (gel). There are two generic variations of the sol-gel technique, one called the colloidal method, the other called the polymeric (or alkoxide) method. Different types of starting materials (precursors) may be used depending on the sol-gel technique, both routes involving suspending or dissolving the precursor(s) in a suitable liquid, usually water for the colloidal route and alcohol for the polymeric route. The precursor is then activated by the addition of an acid (such as hydrochloric acid) or a base (such as potassium hydroxide). The activated precursors then react together to form a network which grows and ages with time and temperature, such as until it is the size of its container. At this point the viscosity of the liquid increases at an exponential rate until gelation occurs, that is, no more flow is observed.

In an embodiment in which the expandable occlusion element is caused to expand upon contact with or exposure to another substance, such as a fluid, the retainer may be configured to allow passage of fluid therethrough. For instance, the retainer may be formed from a fluid pervious and/or porous material to allow the fluid to permeate therethrough to activate the expandable occlusion material to expand. In an example in which the retainer is formed from a liner material, perforations or holes may be formed through the liner material to allow passage of fluid therethrough. For instance, holes may be formed with needles or a small punch, or by virtue of an underlying mandrel or substrate on which the material of the retainer is formed having holes or other regions to which the coating would not be applied or would not adhere.

In accordance with various further principles of the present disclosure, the occlusive effect of the occlusion device is reversible, at least for a temporary amount of time if not permanently. Reduction of the occlusion created by the occlusion device may be desired to reduce the occlusive effect thereof (e.g., to permit reduced passage of materials through the body passage through which the occlusion device is deployed), and/or to allow passage of another device through the occlusion device (e.g., through the lumen of the support structure). In some embodiments, reversing of the occlusive effect of the occlusion device is achieved simply by removing and/or releasing and/or ejecting the expandable occlusion element from its association with the support structure. The expandable occlusion element may be released such as by detaching the structure carrying the expandable occlusion element, or otherwise decoupling the expandable occlusion element from the support structure. Additionally or alternatively, the expandable occlusion element may be released by disrupting the retainer structure holding the expandable occlusion element with respect to the support structure. It will be appreciated that terms such as opening, rupturing, tearing, breaking, etc., and other grammatical forms thereof, may be used interchangeably herein without intent to limit to refer to manners of altering the integrity of the structure of the retainer to allow the expandable occlusion element to be released therefrom.

Additionally or alternatively, expansion of the expandable occlusion element may be reversed by causing the expandable occlusion element to return to its original size or at least to reduce in size, or otherwise deactivating the expandable occlusion element. In some embodiments, the expansion of the expandable occlusion element is reversible so that the expandable occlusion element may contract or otherwise shift to a size smaller than the expanded size, such as upon exposure to an expansion-reversing condition. In some embodiments, reduction of the size of the expandable occlusion element is achieved by exposure or removal of exposure to another element such as liquid (e.g., by exposure to a desiccant) and/or by exposure to a change in temperature or other ambient condition to reverse the expandable phenomenon. In some embodiments, a portion of the support structure and/or retainer may include a conductive material (e.g., a threaded heat-conductive suture) to aid in the transmission of energy, such as heat energy, to the expandable occlusion element to reverse expansion of the expandable occlusion element. In embodiments in which the expandable occlusion material is a hydrogel, the expandable occlusion element may be thermo-sensitive hydrogel beads which can be shrunk in vivo by heating. Examples of such beads include those having a lower critical solution temperature (LCST) above body temperature (37° C.), which will shrink upon heating to a temperature above the LCST. Specific examples of such hydrogels include those based on poly(N-isopropylacrylamide) such as poly(N-isopropylacrylamide-co-N,N-dimethylacrylamide) (PNIPAM-co-DMAAM)), which has a reported LCST around 40° C., poly(N-isopropylacrylamide-b-poly(ethylene glycol)-b-poly(N-isopropylacrylamide (PNIPAM-b-PVP-b-PNIPAM) tri-block copolymers with a reported LCST of 50° C., and 4 arm PNIPAM-b-PEG copolymers with a central PEG and four PNIPAM arms with a reported LCST of 41° C., hydrogels based on poly[2-(dimethylamino)ethyl methacrylate] (PDMAEMA), with a reported LCST of around 50° C., and hydrogels based on poly(N-vinyl caprolactam) such as poly(N-vinyl caprolactam)-b-poly(ethylene glycol)-b-poly(N-vinyl caprolactam) (PNVCl-b-PEG-b-PNVCl) triblock copolymers with a reported LCST 47 to 37° C. (depending on molecular weight).

Various embodiments of occlusion devices will now be described with reference to examples illustrated in the accompanying drawings. Reference in this specification to “one embodiment,” “an embodiment,” “some embodiments”, “other embodiments”, etc., indicates that one or more particular features, structures, and/or characteristics in accordance with principles of the present disclosure may be included in connection with the embodiment. However, such references do not necessarily mean that all embodiments include the particular features, structures, and/or characteristics, or that an embodiment includes all features, structures, and/or characteristics. Some embodiments may include one or more such features, structures, and/or characteristics, in various combinations thereof. Moreover, references to “one embodiment,” “an embodiment,” “some embodiments”, “other embodiments”, etc., in various places in the specification are not necessarily all referring to the same embodiment, nor are separate or alternative embodiments necessarily mutually exclusive of other embodiments. When particular features, structures, and/or characteristics are described in connection with one embodiment, it should be understood that such features, structures, and/or characteristics may also be used in connection with other embodiments whether or not explicitly described, unless clearly stated to the contrary. It should further be understood that such features, structures, and/or characteristics may be used or present singly or in various combinations with one another to create alternative embodiments which are considered part of the present disclosure, as it would be too cumbersome to describe all of the numerous possible combinations and subcombinations of features, structures, and/or characteristics. Moreover, various features, structures, and/or characteristics are described which may be exhibited by some embodiments and not by others. Similarly, various features, structures, and/or characteristics or requirements are described which may be features, structures, and/or characteristics or requirements for some embodiments but may not be features, structures, and/or characteristics or requirements for other embodiments. Therefore, the present disclosure is not limited to only the embodiments specifically described herein, and the examples of embodiments disclosed herein are not intended as limiting the broader aspects of the present disclosure.

It will be appreciated that common features in the accompanying drawings are identified by common reference elements and, for the sake of brevity and convenience, and without intent to limit, the descriptions of the common features are generally not repeated. For purposes of clarity, not all components having the same reference number are numbered. Moreover, similar elements of a group of elements may be indicated by a number and letter, and reference may be made generally to such elements as a group by the number alone (without including the letters associated with each similar element).

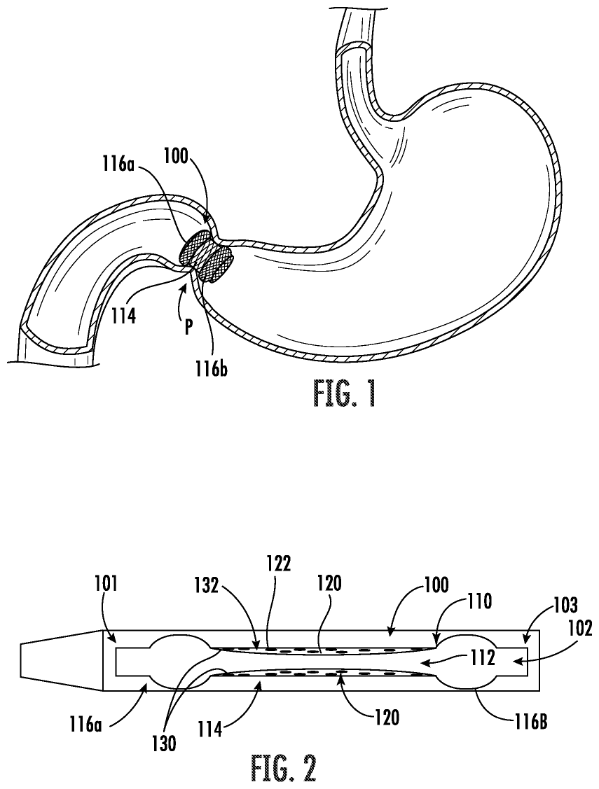

Turning now to the drawings, an example of an embodiment of an occlusion device 100 formed in accordance with various principles of the present disclosure is illustrated in FIG. 1 positioned across a pylorus P as an example of an environment for the occlusion device 100. The occlusion device 100 may be configured to shift between a collapsed delivery configuration, such as illustrated in FIG. 2, and an expanded deployment configuration, such as illustrated in FIG. 1 in which the occlusion device 100 is securely positioned across a body passage such as a pylorus P.

In accordance with various principles of the present disclosure, an occlusion device, such as the occlusion device 100 illustrated in FIG. 1 and FIG. 2, includes a support structure 110 and an expandable occlusion element 120. It will be appreciated that the support structure 110 may be in the form of a stent configured to shift between a collapsed delivery configuration, such as illustrated in FIG. 2, and an expanded deployment configuration, such as illustrated in FIG. 1, such as known or heretofore known in the art, the present disclosure not being limited to a specific structure for the stent. In the illustrated example of an embodiment of an occlusion device 100, the support structure 110 includes a saddle region 114 between at least one retention element 116a on or along a first end 101 (e.g., a distal end) of the support structure 110 and at least one retention element 116b on or along a second end 103 (e.g., a proximal end) of the support structure 110. The retention members 116a, 116b generally extend radially outwardly from the saddle region 114 at or adjacent an end of the saddle region 114. The retention members 116a, 116b may be sized, shaped, configured, and dimensioned to retain the support structure 110 with respect to a deployment site, such as by engaging anatomical structures (e.g., such as walls on either side of the body passage through which the stent extends, as illustrated in FIG. 1). The retention members 116a, 116b preferably are structured to have sufficient retaining strength to withstand anatomical forces acting to displace the occlusion device 100, such as in manners known or heretofore known in the art. In some embodiments, one or both of the retention members 116a, 116b are double-walled retention members, i.e., formed of two walls adjacent or spaced apart from each other. In some embodiments, at least two spaced-apart retention members, each of which may optionally be a double-wall retention member, may extend radially outwardly from each end or one end of the saddle region 114. In embodiments in which the occlusion device 100 is to be placed across a pylorus P, the saddle region 114 is sized and dimensioned to traverse the pyloric region, and generally has a length of approximately 15-20 mm, and diameter, from stenotic condition (in a stenosed passage) to post-surgical arrangement (e.g., a larger passage) of approximately 10 mm-25 mm. The retention members 116a, 116b generally have a diameter larger than the diameter of the saddle region 114 to promote antimigration from the highly motile pyloric region.

The expandable occlusion element 120 of the example of an embodiment of an occlusion device 100 illustrated in FIG. 1 and FIG. 2 is associated with the support structure 110 to adjust the size of the lumen 102 through the occlusion device 100, such as to occlude the lumen 102. The expandable occlusion element 120 may include an expandable occlusion material 122 capable of self-expansion. The expandable occlusion element 120 is shiftable from an initial unexpanded configuration to an expanded configuration which may be considered enlarged, swollen, etc. compared to the initial configuration. Typically, the expandable occlusion element 120 is delivered in its initial configuration with the occlusion device 100 in its delivery configuration (such as illustrated in FIG. 2), and may remain in its initial configuration once the occlusion device 100 has expanded to its deployment configuration (such as illustrated in FIG. 1) until expansion of the expandable occlusion element 120 is desired or medically indicated to reduce the size (e.g., diameter) of the lumen 102 through the occlusion device 100. In some embodiments, the expandable occlusion element 120 is positioned and configured to occlude (e.g., substantially fully occlude) the lumen 102 through the occlusion device 100. The expandable occlusion element 120 may be configured to be returned from its expanded configuration to its initial configuration or at least a reduced configuration relative to the expanded configuration. The expandable occlusion element 120 may be formed of an expandable occlusion material 122, such as a gel or hydrogel. In some embodiments, the expandable occlusion element 120 (e.g., the expandable occlusion material 122) is in the form of beads. As discussed above, the size of the expandable occlusion element 120 may be selected and/or controlled to adjust the size of the lumen 102 of the occlusion device 100 as desired or medically indicated. With a homogenous beading, all hydrogel beads would have generally the same start and finish size (e.g., diameter) ranges. Beads of different sizes may be used. As the beads expand to differing sizes, the ability to efficiently and effectively pack a given region in which the expandable occlusion element is positioned is improved. (smaller beads fitting into spaces where larger beads would not readily fit).

A retainer may be provided to maintain the expandable occlusion element in position with respect to the support structure of an occlusion device formed in accordance with various principles of the present disclosure. The retainer may be in any of a variety of configurations and/or positions with respect to the support structure of the occlusion device. For instance, the retainer may retain the expandable occlusion element with respect to the support structure of the occlusion device along a portion of the occlusion device (cooperating with a structure of the occlusion device), or may independently hold the expandable occlusion element separate from the occlusion device. The retainer may be positioned within, outside, along, or otherwise with respect to the occlusion device.

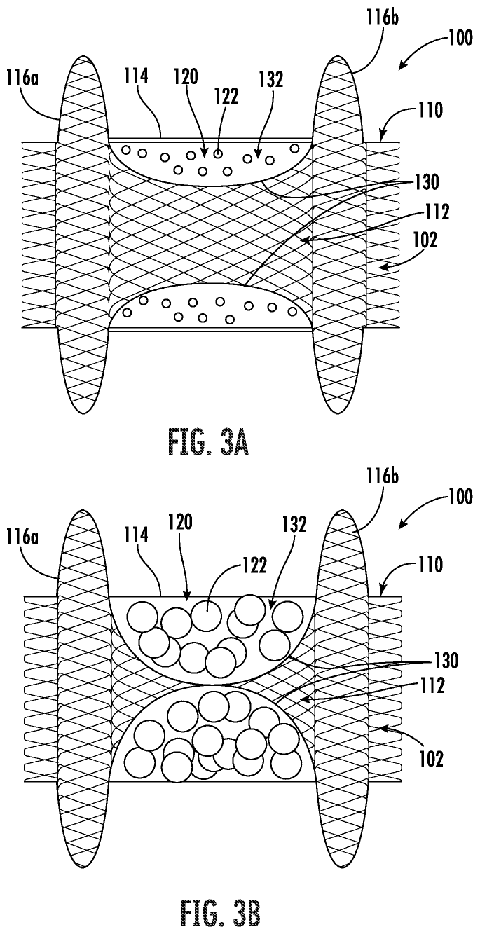

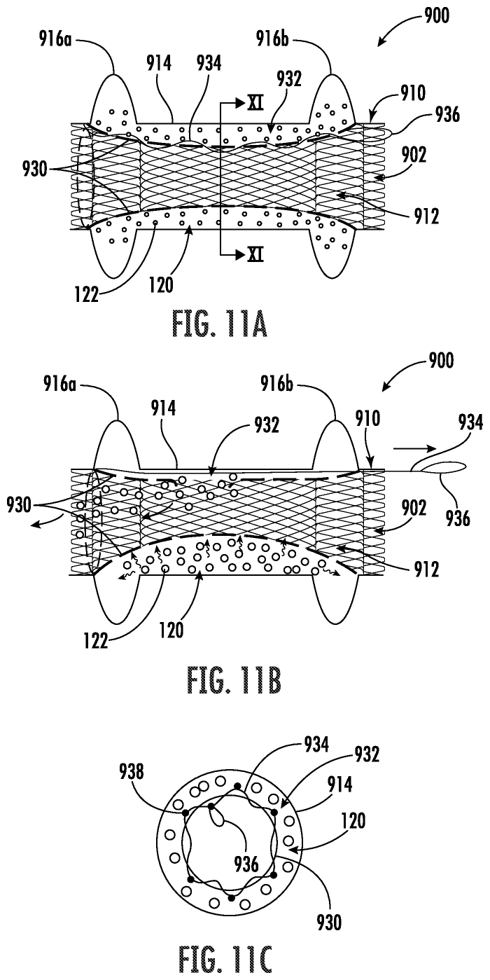

In the example of an embodiment illustrated in FIG. 2, FIG. 3A, and FIG. 3B, a retainer 130 is positioned along the lumen 102 of the occlusion device 100, such as along the lumen 112 of the support structure 110 of the occlusion device 100. The retainer 130 forms a retention area 132 within the lumen 102 of the occlusion device 100, between the retainer 130 and a portion of the support structure 110. The retainer 130 may extend along (e.g., substantially parallel to) the saddle region 114 of the support structure 110. In some embodiments, the retainer 130 extends along the entire length of the saddle region 114 of the support structure 110. However, it will be appreciated that the retainer 130 may, instead, extend along only a portion of the length of the saddle region 114. Optionally, portions of the retention members 116a, 116b may define the retention area 132 with the first retainer 130a and the second retainer 130b as well (such as in the example of an embodiment of an occlusion device 900 illustrated in FIG. 11A and FIG. 11B).

The retainer 130 may be formed as a support element spaced away from the support structure 110. For instance, in some embodiments, the retainer 130 is formed from a liner material used to coat the support structure 110 (e.g., to inhibit or prevent tissue ingrowth), such as known in the art. The retainer 130, if formed from a liner material, may be formed in same manner in which the coating on the support structure 110 is formed, with a spatial separation from the outer coating, so that the retainer 130 is spaced apart from the generally fluid-impermeable tissue-growth-inhibiting coating provided on the exterior of the support structure 110. The wall of the retainer 130 may be loose fitting with respect to the support structure 110 to define the retention area 132 therebetween, such as in the form of a circumferential toroidal pouch. For instance, a mandrel may be positioned within support structure 110 spaced inwardly from the saddle region 114. A coating sprayed over the support structure 110 to coat the support structure 110 (e.g., to prevent tissue ingrowth into the wall of the support structure 110 to allow removability of the support structure 110 from the deployment site if desired or medically indicated, as known in the art) may also be sprayed on the mandrel to form a liner wall spaced inwardly from the wall of the support structure 110 to form the retainer 130.

As illustrated in FIG. 2, the occlusion device 100 may be delivered in a delivery configuration with the retainer 130 in a delivery configuration facilitating delivery of the occlusion device 100 to a deployment site. For instance, in a delivery configuration, the retainer 130 may leave the lumen 102 of the occlusion device 100 unoccluded, such as to permit a guidewire or other delivery device to extend therethrough to facilitate delivery of the occlusion device 100 in a manner known or heretofore known in the art, the present disclosure not being limited in this manner.

Once the occlusion device 100 has been deployed and shifts to its delivery configuration, as illustrated in FIG. 3A, the lumen 102 through the occlusion device 100 may remain unoccluded by the retainer 130 so long as the expandable occlusion element 120 is not activated to expand. However, once the expandable occlusion element 120 is activated to expand, the expandable occlusion element 120 expands into the lumen 102 of the occlusion device 100, shifting the retainer 130 therewith. It will be appreciated that the size or volume, as well as the expansion properties of the expandable occlusion element 120 are selected so that the final expansion configuration of the expandable occlusion element 120 achieves the desired occlusion of the lumen 102 of the occlusion device 100, which may be just a partial occlsuion. For instance, in the example of an embodiment illustrated in FIG. 3B, the expandable occlusion element 120 expands to substantially fully occlude the lumen 102 of the occlusion device 100. However, other configurations in which only partial occlusion is achieved are within the scope of the present disclosure.

As noted above, various configurations and positions of a retainer are within the scope of the present disclosure, examples of which will now be described with reference to the further drawings. In the following description, elements or components similar to those in the example of an embodiment illustrated in FIG. 2, FIG. 3A, and FIG. 3B are designated with the same reference numbers increased by a multiple of 100, and redundant description is omitted for the sake of brevity and convenience. Moreover, certain features in one embodiment may be used across different embodiments and are not necessarily individually labeled when appearing in different embodiments.

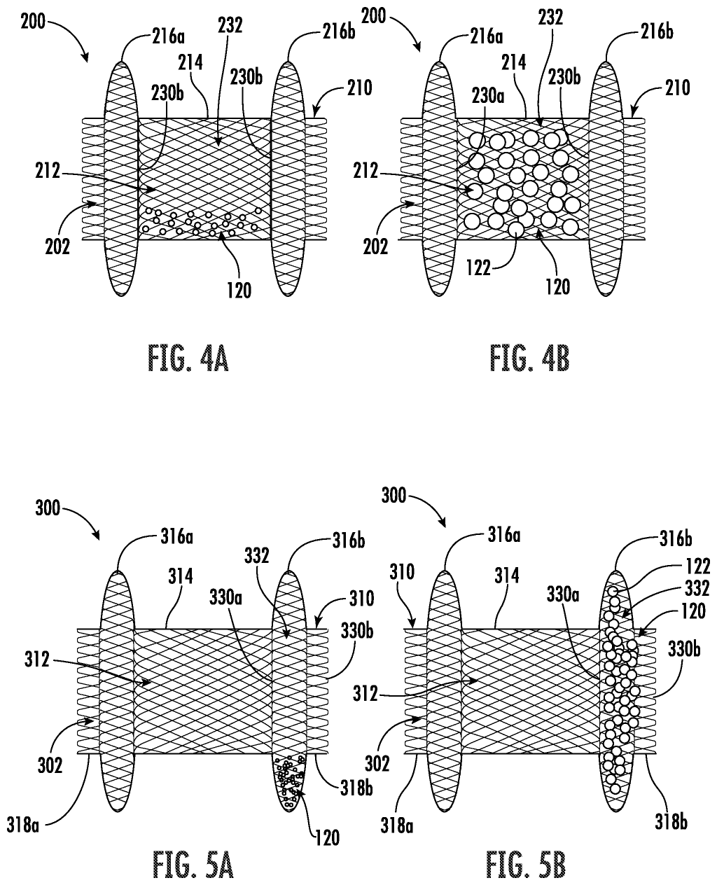

An example of an embodiment of an occlusion device 200 having a support structure 210 and an expandable occlusion element 120 similar to those of the example of an embodiment of FIG. 2, FIG. 3A, and FIG. 3B is illustrated in FIG. 4A and FIG. 4B. Like the example of an embodiment of FIG. 2, FIG. 3A, and FIG. 3B, the example of an embodiment illustrated in FIG. 4A and FIG. 4B also includes a retainer 230 positioned within the lumen 202 of the occlusion device 200. However, the configuration and position of the retainer 230 in the example of an embodiment illustrated in FIGS. 4A and 4B differs from the configuration and position of the retainer 130 in the example of an embodiment illustrated in FIGS. 2, 3A, and 3B. Instead of extending generally along the saddle region 214 of the support structure 210 (as does the retainer 130 of the occlusion device 100 of FIG. 2, FIG. 3A, and FIG. 3B), the retainer 230 of the occlusion device 200 of the example of an embodiment illustrated in FIG. 4A and FIG. 4B is positioned transverse to (e.g., perpendicular to) the longitudinal extent of the occlusion device 200. For instance, the retainer 230 may be positioned transverse to the saddle region 214 of the support structure 210 of the occlusion device 200. As such, the retainer 230 forms or defines a retention area 232 within the lumen 202 of the occlusion device 200 by extending across the lumen 202 of the occlusion device 200. The retention area 232 may be considered to be defined by a first retainer 230a, a second retainer 230b positioned spaced apart from the first retainer 230a, and portions of the support structure 210 extending therebetween. The first retainer 230a and the second retainer 230b may be substantially parallel to each other or inclined with respect to each other without detracting from the basic principles of the present disclosure. At least a portion of the saddle region 214 of the support structure 210 defines, with the first retainer 230a and the second retainer 230b, the retention area 232 between the first retainer 230a and the second retainer 230b. Optionally, the entire length of the saddle region 214 may define the retention area 232 with the first retainer 230a and the second retainer 230b.

When the occlusion device 200 is initially expanded (e.g., from a delivery configuration such as illustrated in FIG. 2) into a deployed configuration, as illustrated in FIG. 4A, and before the expandable occlusion element 120 has been expanded, the lumen 202 through the occlusion device 200 (and the lumen 212 through the support structure 210) may not be fully obstructed by the expandable occlusion element 120 so that elements (e.g., fluids, devices, etc.) may pass therethrough. However, once the expandable occlusion element 120 has been activated to expand, as illustrated in FIG. 4B, the lumens 202, 212 may be partially or fully occluded (as desired or medically indicated) by the expanded expandable occlusion element 120 therein. The expanded expandable occlusion element 120 may create a generally solid mass within the occlusion device 200 which may increase the pull-out force required to move the occlusion device 100 from the deployment site.

It will be appreciated that a retainer extending transverse to the longitudinal extent of a lumen through an occlusion device (such as in the occlusion device 200 of the example of an embodiment illustrated in FIG. 4A and FIG. 4B) may be positioned at locations along the length of the occlusion device other than as in the occlusion device 200. For instance, in the example of an embodiment illustrated in FIG. 5A and FIG. 5B, an occlusion device 300 has a retainer 330 transverse to the longitudinal extent of the occlusion device 300 and positioned with respect to at least one retention member 316b of the support structure 310 of the occlusion device 300 to form a retention area 332 within the retention member 316b. In the illustrated example of an embodiment of a support structure 310 with a double-walled retention member 316b, a first retainer 330a is positioned between the retention member 316b and the saddle region 314 and a second retainer 330b is positioned spaced from the first retainer 330a and further away from the saddle region 314 to form a retention area 332 within the retention member 316b. In some embodiments, such as illustrated, one or both retention members 316a, 316b have a respective extension or lip 318a, 318b which may respectively extend from the associated retention member 316a, 316b in a direction away from the saddle region 314 and may facilitate grasping of the support structure 310 (such as to remove the support structure 310), in a manner known in the art. The second retainer 330b may be positioned along a portion of the lip 318a, 318b associated with the retention member 316a, 316b along or adjacent which the first retainer 330a is positioned. As such, the retention area 332 may be considered a pouch within a retention member 316 of the occlusion device 300 which may be expanded in size or at least bolstered upon expansion of the expandable occlusion element 120 therein, such as illustrated in FIG. 5B. The expanded expandable occlusion element 120 within the retention member 316b of the occlusion device 300 may increase the pull-out force required to move the occlusion device 300 from the deployment site. For instance, the expanded expandable occlusion element 120 may form a generally solid mass within the retention member 316b, reducing the likelihood or potential of the retention member 316b pulling through the body passage through which the occlusion device 300 has been deployed, such as by resisting bending or collapse of the retention member 316b. In the example of an environment for an occlusion device formed in accordance with various principles of the present disclosure illustrated in FIG. 1, the retention member 316b may be a proximal retention member 316b strengthened upon expansion of the expandable occlusion element 120 to resist being dislodged and passing through the pylorus P and into the duodenum.

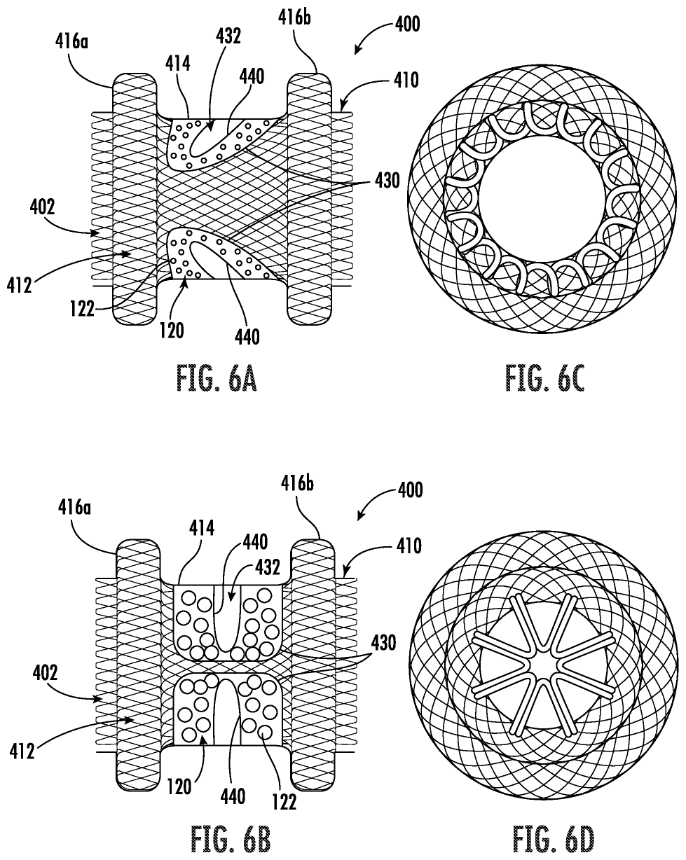

In accordance with various principles of the present disclosure, an expandable occlusion element within a retainer positioned within an occlusion device formed in accordance with various principles of the present disclosure may interact with an element of a support structure of the occlusion device. For instance, in an example of an embodiment of an occlusion device 400 as illustrated in FIG. 6A and FIG. 6B, the occlusion device 400 may include a support structure 410 having one or more selectively occlusive structures 440 extending within the retention area 432 formed by a retainer 430. The retainer 430 also encloses within the retention area 432 an expandable occlusion element 120 between the retainer 430 and a structure of the support structure 410 (e.g., the saddle region 414). The selectively occlusive structures 440 may be in the form of one or more flexible loops or other movable structures formed from or coupled to the support structure 410 and which is shiftable from a deployment, relaxed, configuration to an occlusion configuration. In the deployment configuration, the occlusive structures 440 generally extends along (e.g., folded or otherwise laying across) the support structure 410, such as along the saddle region 414 thereof, as illustrated in FIG. 6A, and the end view thereof in FIG. 6C. In such configuration, the selectively occlusive structures 440 enable the occlusion device 400 to be delivered in a compact delivery configuration without interference of the selectively occlusive structures 440. Once the occlusion device 400 is deployed, and the expandable occlusion element 120 expanded, the selectively occlusive structures 440 may shift to an occlusion position generally transverse to the support structure 410, as illustrated in FIG. 6B, and the end view thereof in FIG. 6D. The selectively occlusive structures 440 may form a gated section across the lumen 412 of the support structure 410 and thus across the lumen 402 of the occlusion device 400 to occlude the lumens 402, 412.

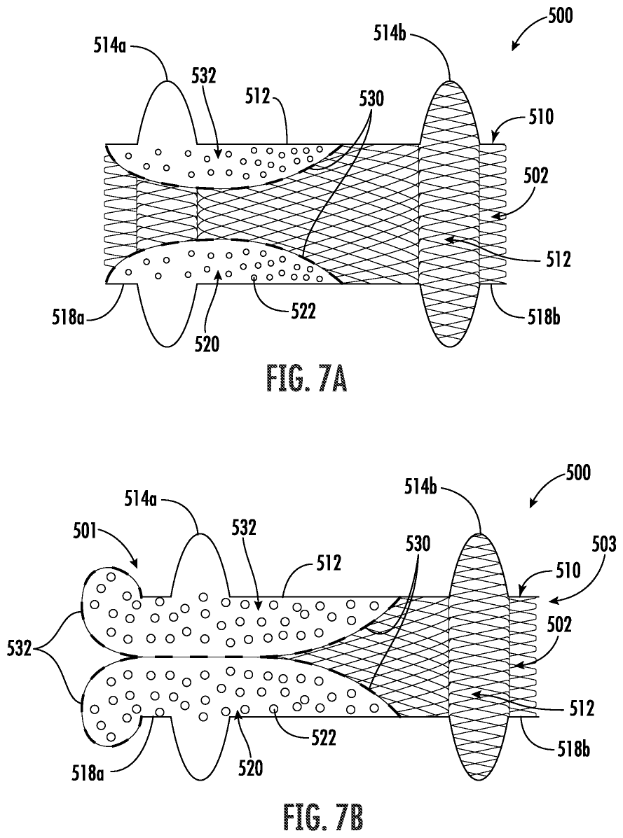

It will be appreciated that although a retainer may be positioned with an occlusion device formed in accordance with various principles of the present disclosure, the retainer and/or the associated expandable occlusion element need not remain completely within the interior of the occlusion device when the expandable occlusion element expands. For instance, in an example of an embodiment of an occlusion device 500 as illustrated in FIG. 7A and FIG. 7B, the expandable occlusion element 120 may expand the retainer 530 such that the retention area 532 extends out one or both of ends 501, 503 of the occlusion device 500, such as beyond one or both of the retention members 516a, 516b, and optionally beyond a lip 518a, 518b, if provided. Such extension of the expandable occlusion element 120 may provide further occlusion of the lumen 502 of the occlusion device 500 than provided by simply an internal occlusion as in the previously described examples of embodiments, such as by providing more solid mass to occlude the lumen 502. It will be appreciated that although the retainer 530 is illustrated in FIG. 7B as exuding out of a distal end 501 (e.g., a distal end), the retainer 530 may additionally or alternatively exude out of a proximal end 503.

Instead of a retainer being formed within an occlusion device formed in accordance with various principles of the present disclosure, a retainer formed in accordance with various principles of the present disclosure may be positioned around or along or about or on (such terms being used interchangeably herein without intent to limit) the exterior of the occlusion device. An externally positioned retainer may be configured to constrict the occlusion device to occlude the lumen therethrough. For instance, the retainer may include an expandable occlusion element which expands inwardly towards the lumen through the occlusion device to restrict or occlude the lumen.

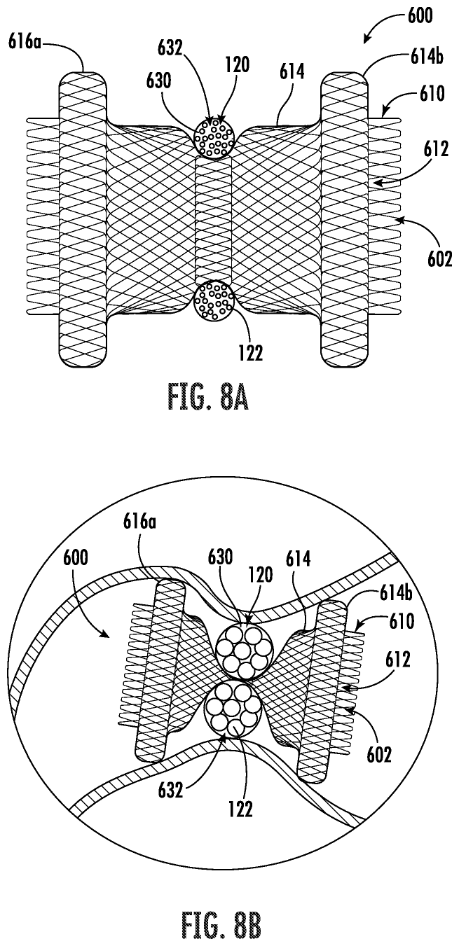

An example of an embodiment of a retainer 630 positioned external to an occlusion device 600 formed in accordance with various principles of the present disclosure is illustrated in FIG. 8A and FIG. 8B. The retainer 630 may be in the form of a band or other structure positioned about the exterior of the occlusion device 600 to retain the expandable occlusion element 120 in place between the retainer 630 and the exterior of the occlusion device 600. In an example of an embodiment illustrated in FIG. 8A, when the occlusion device 600 is in a deployment configuration before the expandable occlusion element 120 has been expanded, the lumen 602 through the occlusion device 600 remains at least partially unoccluded. Once expanded, the expandable occlusion element 120 extends radially inwardly, as illustrated in FIG. 8B, and occludes, or at least partially occludes, the lumen 602 through the occlusion device 500.

In accordance with various principles of the present disclosure, a retainer may form a retention area for an expandable occlusion element independently from the occlusion device. In some embodiments, the retainer 630 of the example of an embodiment of an occlusion device 600 illustrated in FIG. 8A and FIG. 8B may be formed independently of the support structure 610. For instance, the retainer 630 may enclose and retain the expandable occlusion element 120 independently of any structure of the support structure 610, yet be associated with the support structure 610 to reduce the lumen 612 through the support structure 610 upon expansion of the expandable occlusion element 120. In other words, a portion of the support structure 610 does not cooperate with the retainer 630 to enclose the expandable occlusion element 120 within a retention area 632 formed by the retainer 630 and the support structure 610, such as in other embodiments disclosed herein (such as the above-described embodiments).

In some aspects, a retainer which is formed separately and independently from the occlusion device to form a separately formed retention area enclosing the expandable occlusion element independently of the occlusion device may be positioned within the lumen of an occlusion device, rather than being positioned along the exterior of the occlusion device. Such separately formed retainer may be delivered with the occlusion device to the deployment site (e.g., within the occlusion device, or external to the occlusion device such as around or proximal or distal to the occlusion device), or may be delivered separately, such as once the occlusion device has been securely deployed and seated at the deployment site.

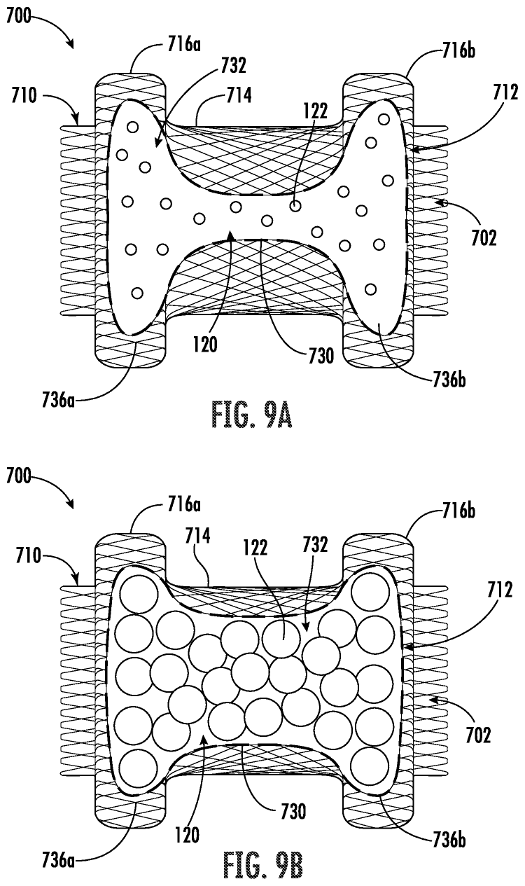

Another example of an embodiment of a retainer 730 separately formed from an example of an embodiment of an occlusion device 700 formed in accordance with various principles of the present disclosure is illustrated in FIG. 9A and FIG. 9B. Because the retainer 730 forms the retention area 732 for the expandable occlusion element 120 independently of the structure of the support structure 710 of the occlusion device 100, the retainer 730 may be deployed separately, after the support structure 710 has been deployed at a deployment site. Thus, the retainer 730 is capable of being deployed within and with the occlusion device 700, or of being positioned and deployed within the occlusion device 700 after the occlusion device 700 has been positioned at a deployment site. When the retainer 730 is positioned within the lumen 702 of the occlusion device 700 in an initial configuration, such as illustrated in FIG. 9A, the walls of the retainer 730 are spaced inwardly from the walls of the support structure 710, allowing materials (e.g., fluids, devices, etc.) to pass through the lumen 702 of the occlusion device 700. As may be appreciated, the retainer 730 fully encloses the expandable occlusion element 120 within a retention area 732, and, in its initial configuration, has a smaller configuration than the expanded configuration illustrated in FIG. 9B. When the expandable occlusion element 120 is expanded, as illustrated in FIG. 9B, the retainer 730 expands, such as to fill or substantially fill the lumen 702 of the occlusion device 700, such as to occlude or at least partially occlude the lumen 702 of the occlusion device 700. In the illustrated example of an embodiment, the retainer 730 has retention members 736a, 736b configured to engage with (e.g., interact, interlock, fit within, etc.) the retention members 716a, 716b of the support structure 710 such that the retainer 730 is held in place with respect to the support structure 710 to resist migration and possible separation from the support structure 710.

It will be appreciated that a separately formed retainer formed in accordance with various principles of the present disclosure may be delivered with, but separate from (e.g., external to), the support structure of an occlusion device formed in accordance with various principles of the present disclosure. The retainer may be moved into a closer position with respect to/in association with the occlusion device upon deployment of the occlusion device at a deployment site. In some embodiments, the retainer may be configured with respect to the support structure such that positioning or movement of the retainer into the lumen of the support structure occurs upon deployment of the occlusion device, such as a result of deployment.

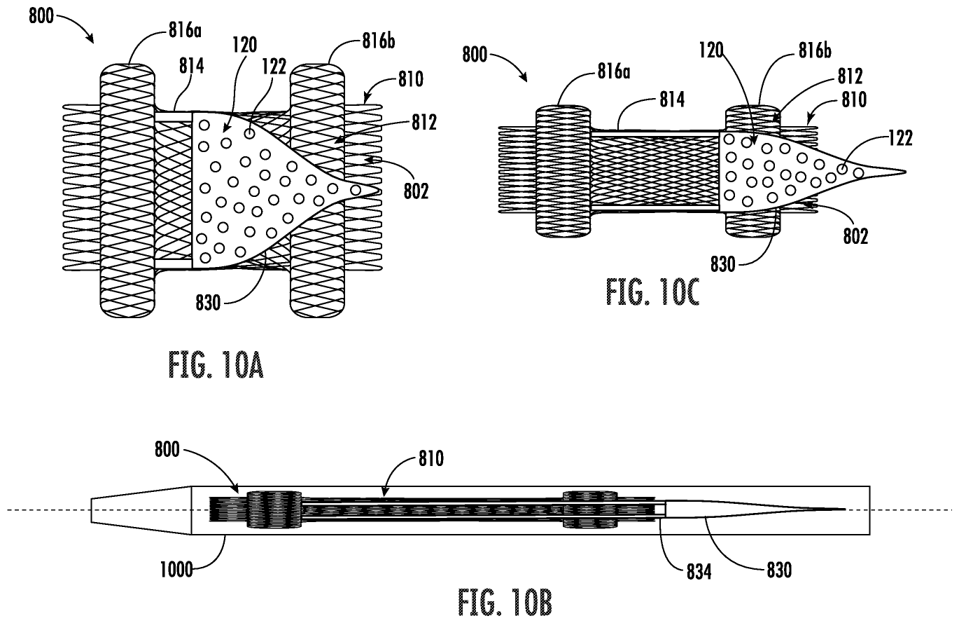

An example of an embodiment of an occlusion device 800 with a separately formed retainer 830 delivered separately from a support structure 810 is illustrated in FIG. 10A, FIG. 10B, and FIG. 10C. As illustrated in FIGS. 10B, rather than being deployed within or around a support structure 810, the retainer 830 trails (is proximal to) the support structure 810 of the illustrated example of an embodiment of an occlusion device 800. It will be appreciated that the retainer 830 may lead (be delivered first/distal to) the support structure 810 instead, without departing from principles of the present disclosure.

In some embodiments, the retainer 830 may be coupled to the support structure 810 to be delivered therewith. For instance, in the example of an embodiment illustrated in FIG. 10B, one or more pull elements 834 (e.g., wires, strings, cords, suture threads, etc.) may couple the retainer 830 and the support structure 810 during delivery of the occlusion device 800. Upon deployment, as the occlusion device 800 shifts to a deployed configuration, such as illustrated in FIG. 10C, the occlusion device 800 expands radially and contracts longitudinally from its delivery configuration (such as illustrated in FIG. 10C) and draws the pull elements 834 and the retainer 830 into the support structure 810, as illustrated in FIG. 10A. In some embodiments, the diameter of the support structure 810 in the delivery configuration may be approximately 3 mm, whereas the diameter of the support structure 810 in the deployment configuration may be approximately 20-30 mm, such increase allowing any slack in the pull elements 834 be taken up within the support structure 810. Additionally or alternatively, the retainer 830 may be formed of a shape memory material having a degree of memory to facilitate being pulled into the support structure 810 upon deployment of the occlusion device 800.

In accordance with various principles of the present disclosure, it may be desirable to permit materials to flow through an occlusion device formed in accordance with various principles of the present disclosure at some point in time after an expandable occlusion element has occluded the lumen through the occlusion device. It will be appreciated that as referenced herein, “materials” which may pass through the lumen of an occlusion device formed in accordance with various principles of the present disclosure include, without limitation, fluids, particles (e.g., chyme), tools, instruments, devices, etc. Various structures and configurations may be implemented to facilitate removal of the expandable occlusion element and/or return of the expandable occlusion element to an unexpanded configuration (or at least a configuration reduced from the expanded configuration causing occlusion of the occlusion device). In some embodiments, return of an occlusion device formed in accordance with various principles of the present disclosure to an unoccluded configuration is achieved with a structure which alters the expandable occlusion element and/or the retainer associated therewith. For instance, a structure may be provided to release the expandable occlusion element from the occlusion device, such as by releasing the expandable occlusion element from the retainer, or by releasing the expandable occlusion element with the retainer and from the support structure of the occlusion device. In some embodiments, the retainer of an occlusion device formed in accordance with various principles of the present disclosure may be formed with a disruptor element configured to permit a controlled manner of disrupting the retainer to release an expandable occlusion element retained therein. Additionally or alternatively, a structure may be introduced to return the expandable occlusion element to an unexpanded configuration.

An example of an embodiment of an occlusion device 900 with a retainer 930 having a disruptor element 934 structured and positioned to interact with the retainer 930 to cause physical disruption of the retainer 930 allowing the expandable occlusion element 120 to be released therefrom, is illustrated in FIG. 11A, FIG. 11B, and FIG. 11C. The disruptor element 934 may be manipulated or moved relative to the retainer 930 to disrupt the structural integrity of the retainer 930. In some embodiments, the disruptor element 934 is provided with grasping feature 936 (e.g., a loop at an end of the disruptor element 934) to facilitate engagement with and pulling of the disruptor element 934 to manipulate or actuate the disruptor element 934 to disrupt the retainer 930. In some embodiments, the disruptor element 934 includes a suture or filament or wire (such elements being referenced interchangeably herein without intent to limit) having a configuration to facilitate disruption of the retainer 930 upon movement of the disruptor element 934 relative to the retainer 930. As illustrated in FIG. 11C, the disruptor element 934 may be passed or tacked from inside to outside the inner wall of the retainer 930 (e.g., as a continuous stitch) in a radial manner. In some embodiments, the disruptor element 934 passes repeatedly from inside to outside the retainer 930 in a radial manner around the circumference of the retainer 930. Pulling of the disruptor element 934 causes the disruptor element 934 to tear the retainer 930 to allow the expandable occlusion element 120 to be released from the retention area 932 within the retainer 930. The disruptor element 934 may be removed once the retainer 930 has been breached and the expandable occlusion element 120 released, or may be allowed to pass naturally through the body (such as, in some embodiments, with the expandable occlusion element 120). Alternative embodiments of configurations of a disruptor element 934 are within the scope and spirit of the present disclosure. For instance, multiple disruptor elements 934, such as multiple sutures or filaments or wires, may be used. In some embodiments, enlarged regions may be formed on the disruptor element 934 such as along the length of the disruptor element 934, such as illustrated in FIG. 11C. Such enlarged regions may cause larger voids in the retainer 930 as the disruptor element 934 ruptures the retainer 930. Such enlarged regions may be formed in a variety of manners, such as knotted sections on the outer areas of the suture line, a beading component threaded into place during manufacture, another component formed separately from the disruptor element and applied or otherwise coupled thereto (e.g., in liquid form which upon application to the disruptor element 934 solidifies, or a solid component otherwise coupled to the disruptor element 934), etc.

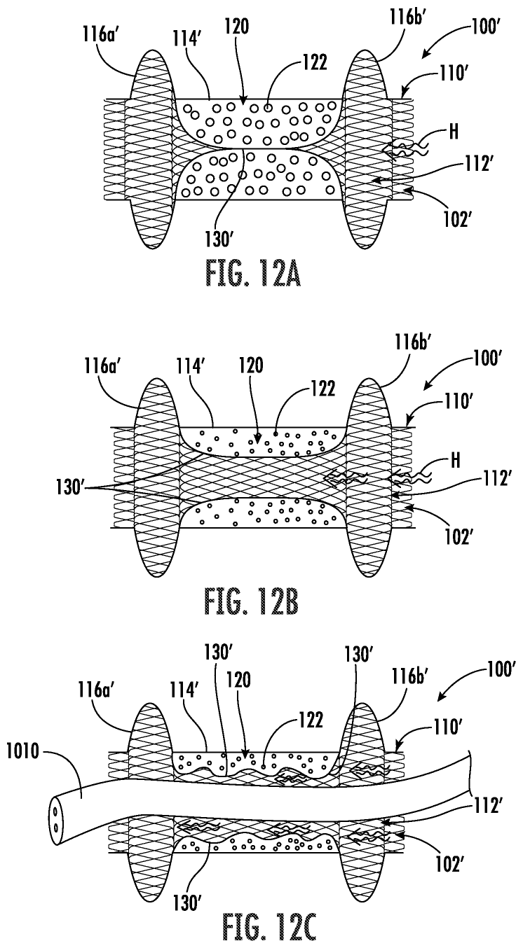

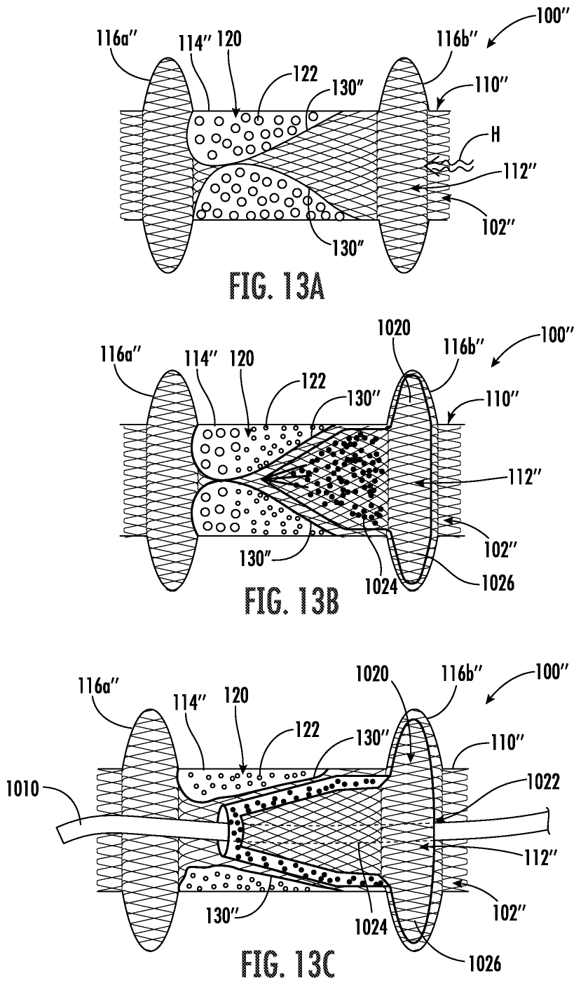

Instead of releasing the expandable occlusion element 120 from the occlusion device, the expandable occlusion element may be triggered or activated to return from an expanded configuration to an unexpanded configuration or at least a sufficiently reduced configuration to allow materials to pass through the lumen of the occlusion device. In some embodiments, a separate element may be inserted adjacent the expandable occlusion element to cause or induce the expandable occlusion element to return from an expanded configuration to an unexpanded configuration (either to the original configuration or at least a less-expanded configuration). A lumen may be provided through such separate element to facilitate passage of an instrument therethrough and through the lumen of the occlusion device. Examples of embodiments of occlusion devices with expandable occlusion elements being shifted from an expanded configuration to an unexpanded configuration are illustrated in FIG. 12A, FIG. 12B, FIG. 12C, FIG. 13A, FIG. 13B, FIG. 13C, as will now be described in further detail.

An example of an embodiment of an occlusion device 100′ similar to the occlusion device 100 illustrated in FIG. 3B is illustrated in FIG. 12A with the expandable occlusion element 120 associated therewith in an expanded configuration, and in FIG. 12B with the expandable occlusion element 120 shifting to a reduced-sized configuration. In the illustrated example of an embodiment, such shifting is initiated/effected by application of energy such as heat, indicated schematically by heat arrows H. Once the expandable occlusion element 120 has been sufficiently contracted in size so that the lumen 102′ through the occlusion device 100′ is not completely occluded, an ancillary device 1010 may be extended through the lumen 102′ of the occlusion device 100′. The ancillary device 1010 may be any additional device, instrument, tool, etc., which may be used in or around the deployment site for the occlusion device 100, such as known to those of ordinary skill in the art. Additionally or alternatively, other materials (e.g., chyme) may be allowed to pass through the lumen 102′ of the occlusion device 100′. It will be appreciated that once the mechanism effecting reduction of the size of the expandable occlusion element 120 is removed (e.g., heat H is no longer applied to the expandable occlusion element 120), the expandable occlusion element 120 may be allowed to return to its fully expanded configuration if desired. It will be appreciated that instead of heat, another energy form, such as ultrasonics, may be applied to reduce the size of the expanded expandable occlusion element 120.

In some embodiments, as illustrated in FIG. 13A, FIG. 13B, FIG. 13C, the mechanism effecting reduction of the size of the expandable occlusion element 120 is a separate structural device, such as a secondary scaffold 1020 configured to deliver an element to the expandable occlusion element 120 which causes reduction of the size of the expandable occlusion element 120. It will be appreciated that the element may be an environmental change (e.g., heat) or a structure having a size-reducing effect on the expandable occlusion element 120. For instance, the secondary scaffold 1020 may achieve an environmental change by delivering an exothermic element sufficiently adjacent to the expandable occlusion element 120 to effect a reduction in the size of the expandable occlusion element 120. Additionally or alternatively, the secondary scaffold 1020 may be configured to deliver (e.g., by containing or carrying) a dessicant (sorbitol or mannitol) for a water-based expandable occlusion element 120 (e.g., a hydrogel) which osmotically encourages fluid in the expandable occlusion element 120 to exit to reduce the size of the expandable occlusion element 120. The retainer 130′ may be configured, as illustrated in FIG. 13A and FIG. 13B, to have a taper or otherwise to receive the secondary scaffold 1020. Exposure and/or contact of the expandable occlusion element 120 with the secondary scaffold 1020 causes the expandable occlusion element 120 to reduce in size and/or the retainer 130′ (with the expandable occlusion element 120 therein) to become more pliable to allow passage of materials such as an ancillary device 1010 through the lumen 102′ of the occlusion device 100′, as illustrated in FIG. 13C.

In accordance with various principles of the present disclosure, a mechanism of action of any of the examples of occlusion devices described above may be follows. The occlusion device may be advanced to the deployment site using standard endoscopic techniques and then deployed across the pylorus P, such as illustrated in FIG. 1. The occlusion device, in situ, is allowed to be exposed to an element which causes the expandable occlusion element associated therewith to expand. For instance, the natural liquid surrounding the occlusion device or an artificially-introduced fluid (e.g., a water infusion), such as delivered by an endoscopic system (e.g., the system used to introduce/deliver the occlusion device), may cause expansion of the expandable occlusion element. The expandable occlusion element expands upon/after exposure, taking up the space within the retention area within the retainer such that the inner lumen of the occlusion device becomes blocked, such as illustrated in FIG. 3B, FIG. 4B, FIG. 5B, FIG. 6B, FIG. 7B, FIG. 8B, FIG. 9B, FIG. 10C, FIG. 11A, FIG. 12A. The expansion outwards is contained by the support structure and generally does not impact the device placement.

An occlusion device formed in accordance with one or more of the above-described principles may allow for one or various benefits, such as, without limitation, a low-profile device deliverable with an expandable intermediary device; a single intervention to place the device and a “natural” closure; option for a remote, non-invasive assessment of occlusion efficacy (e.g., through barium/fluoroscopic assessment); reversibility; and other appreciable benefits.

Various structures and features of the embodiments described herein and illustrated in the figures have several separate and independent unique benefits. In view of the above, it should be understood that the various embodiments illustrated in the figures have several separate and independent features, which each, at least alone, has unique benefits which are desirable for, yet not critical to, the presently disclosed occlusion device. Therefore, the various separate features described herein need not all be present in order to achieve at least some of the desired characteristics and/or benefits described herein. It will further be appreciated that various features described with respect to one embodiment may be applied to another embodiment, whether or not explicitly indicated. Only one of the various features may be present in an occlusion device formed in accordance with various principles of the present disclosure. Alternatively, one or more of the features described with reference to one embodiment can be combined with one or more of the features of any of the other embodiments provided herein. That is, any of the features described herein can be mixed and matched to create hybrid designs, and such hybrid designs are within the scope of the present disclosure.

Therefore, the present invention is not limited to only the embodiments specifically described herein. The above descriptions are of illustrative examples of embodiments only, and are not intended as limiting the broader aspects of the present disclosure.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An occlusion device comprising:
a support structure having a lumen defined therethrough; and
an expandable occlusion element formed separately from the support structure and held with respect to the support structure, wherein the expandable occlusion element is expandable with respect to the support structure to occlude the support structure lumen.

2. The occlusion device of claim 1, wherein the expandable occlusion element is formed from a self-expanding material.

3. The occlusion device of claim 2, wherein the expandable occlusion element is formed from a hydrogel.

4. The occlusion device of claim 2, wherein the expandable occlusion element is held within a retainer associated with the support structure.

5. The occlusion device of claim 4, wherein the retainer is formed of a porous or permeable material.

6. The occlusion device of claim 1, wherein the expandable occlusion element is held within a retainer associated with the support structure.

7. The occlusion device of claim 6, wherein the retainer includes a disruptor element configured to disrupt the integrity of the retainer to release the expandable occlusion element therefrom.

8. The occlusion device of claim 6, wherein the retainer is separately formed from the support structure.

9. The occlusion device of claim 8, wherein the retainer is deployed separately from the support structure.

10. The occlusion device of claim 9, wherein the support structure is configured to shift between an unexpanded deployment configuration and an expanded deployment configuration, and to draw the retainer into the support structure lumen upon expanding into the deployment configuration.

11. The occlusion device of claim 1, wherein expansion of the expandable occlusion element is reversible to allow passage of materials through the support structure lumen.

12. The occlusion device of claim 11, further comprising a secondary scaffold configured to deliver an element to the expandable occlusion element which causes reduction of the size of the expandable occlusion element.

13. A system for occluding a body passage, the system comprising:
a delivery device; and
an occlusion device, the occlusion device having a support structure with a lumen defined therethrough, and an expandable occlusion element formed separately from the support structure and held with respect to the support structure, wherein the expandable occlusion element is expandable with respect to the support structure to occlude the support structure lumen.

14. The system of claim 13, wherein the expandable occlusion element is formed from a self-expanding material.

15. The system of claim 14, wherein the expandable occlusion element is formed from a hydrogel.

16. The system of claim 13, wherein the expandable occlusion element is held within a retainer associated with the support structure.

17. A method of occluding a body passage, the method comprising:
delivering an occlusion device to a body passage in a delivery configuration;
deploying the occlusion device so that a support structure of the occlusion device shifts to an expanded deployment configuration; and
causing an expandable occlusion element formed separately from the support structure to expand to at least partially occlude a lumen extending through the support structure.

18. The method of claim 17, wherein causing the expandable occlusion element to expand comprises exposing the expandable occlusion element to an expansion-inducing condition.

19. The method of claim 17, further comprising reversing the expansion of the expandable occlusion element to allow materials to pass through the support structure lumen.

20. The method of claim 17, further comprising release of the expandable occlusion element from the support structure to allow materials to pass through the support structure lumen.
